(19) United States
(12) Patent Application Publication
(10) Pub. No.: US 2009/0074761 A1
(43) Pub. Date: Mar. 19, 2009

(54) Therapeutic beta-glucan combinations

(76) Inventor: Jun Yan, Louisville, KY (US)

Correspondence Address: BIOPOLYMER ENGINEERING , INC. DBA BIOTHERA , INC, C/O INTELLEVATE , LLC, P.O. BOX 52050, MINNEAPOLIS, MN, 55402, US

(21) Appl. No.: 12/150,049

(22) Filed: Apr. 24, 2008

Related U.S. Application Data
(60) Provisional application No. 60/913,679, filed on Apr. 24, 2007.

(51) Int. Cl. A61K 31/716; A61P 35/00; A61K 39/395; A61K 31/704

(52) U.S. Cl. 424/133.1; 514/54; 424/130.1; 424/141.1; 514/34

(57) ABSTRACT

A therapeutic composition for treating a proliferative disorder includes a VEGF antagonist and β-glucan. VEGF is overexpressed in some tumor types. The efficacy of treatment with VEGF antagonists capable of activating complement in combination with β-glucan is significantly increased.

### GOVERNMENT SUPPORT

The invention was supported in whole or in part by a grant NIH RO1 CA86412 from the National Cancer Institute. The Government has certain rights in the invention.

### BACKGROUND OF THE INVENTION

This application claims the benefit of U.S. Provisional Application No. 60/913,679, entitled THERAPEUTIC B-GLUCAN COMBINATIONS, filed Apr. 24, 2007.

The present invention relates to therapeutic compositions for treatment of cancer. More particularly, the present invention relates to protein antagonists of mVEGF in combination with β-glucan and their use in enhancing anti-proliferative immunotherapy.

Lung cancer continues to be the number one cancer-related mortality in the United States for both men and women. 213,380 new cases of lung cancer and 160,390 deaths are expected in 2007. In Kentucky, 3,450 deaths are expected from lung cancer this year—over a third of all cancer related deaths (Jemal et al., CA Cancer J Clin 55: 10-30).

Lung cancer is divided in two major classes: small cell (SLC) and non-small cell types (NSCLC). NSCLC accounts for 80-85% of all lung cancer cases and is subdivided in three main types: adenocarcinoma, squamous cell and large-cell carcinoma. Adenocarcinoma is now the predominant histological subtype in many countries including the United States. The 5-year survival for advanced stage, metastatic and recurrent NSCLC is estimated to be only 1-7%. Over the last decade, biological agents and targeting molecules, such as monoclonal antibodies (mAb), involved in tumor growth and progression have been developed. Despite those progresses, however, antibody (Ab) therapy is not uniformly effective. Developing novel strategies to maximize the efficacy of anti-tumor mAbs is necessary to overcome this limitation in cancer therapy.

β-glucan is a complex carbohydrate derived from sources including yeast and other fungi, bacteria and cereal grains. β-glucans are biological response modifiers (BRMs) and have existed for centuries in Asian traditional medicine. They have been used to treat malignancy clinically (with varying and unpredictable success) for decades, particularly in Japan.

Previous in vitro studies demonstrated that soluble yeast β-glucan, such as Imprime PGG™, bound to a lectin domain within the COOH-terminal region of the CD11b subunit of complement receptor 3 (CR3, CD11b/CD18, αₘβ₂ integrin, Mac-1) (Thorton et al., J Immunol 156:1235-1246). Studies have indicated that β-glucans prime neutrophils, macrophages (Mφ) and NK cells for cytotoxicity against tumors opsonized with iC3b as a result of complement activation by anti-tumor mAbs or natural Abs (Vetvicka et al., J Clin Invest 98:50-61; Yan et al., Expert Opin Biol Ther 5:691-702). Dual ligation of neutrophil CR3 mediated by the I-domain ligand, iC3b, and the lectin-like domain (LLD) ligand, β-glucan, leads to degranulation and cytotoxic responses. Thus, β-glucan-mediated tumor immunotherapy utilizes a novel mechanism by which innate immune effector cells are primed to kill iC3b-opsonized tumor cells.

### SUMMARY OF THE INVENTION

The present invention is a therapeutic composition to a proliferative disorder. The therapeutic composition includes a VEGF antagonist and β-glucan. The present invention also encompasses treatments and kits utilizing the therapeutic compositions.

### BRIEF DESCRIPTION OF THE DRAWINGS

FIG. 1A is a graphical representation of flow cytometry results indicating the presence of membrane-bound VEGF on SKOV-3 cells.

FIG. 1B shows microscopic images of VEGF expression on SKOV-3 tumor cells.

FIG. 1C is a graphical representation showing membrane bound VEGF on SKOV-3 cells.

FIG. 2 graphically shows CR3-dependent cellular cytotoxicity mediated by β-glucan and anti-tumor mAbs.

FIGS. 3A and 3B are graphic representations of the tumoricidal activity of β-glucan and humanized anti-VEGF mAb.

FIGS. 4A-4C are graphic representations of the tumoricidal activity of β-glucan and anti-VEGF mAb.

FIG. 5A shows microscopic images of iC3b deposition in SKOV-3 tumors.

FIG. 5B shows microscopic images of neutrphil infiltration in SKOV-3 tumors.

FIG. 6 shows microscopic images of tumor blood vessel development after anti-VEGF mAb therapy.

### DETAILED DESCRIPTION OF THE INVENTION

Tumor immunotherapy with humanized mAbs is now accepted clinical practice. Examples of such mAbs include Herceptin™ (trastuzumab) and Rituxan™ (rituximab) for patients with Her-2/neu⁺ metastatic breast mammary carcinoma and B cell lymphoma, respectively, and Erbitux™ (cetuximab) for patients with over-expressed EGFR colon or rectal cancers.

Vascular endothelial growth factor (VEGF) stimulates abundant angiogenesis, which allows exponential tumor growth and provides the hematogenous route for metastasis (Rini et al., J Clin Oncol 23:1028-43; Fidler et al., Cell 79:185-8). VEGF-A is the member of the VEGF family that seems to exercise the greatest control of angiogenesis during tumor and metastatic development (Senger et al., Science 219:983-5; Leung et al., Science 246:1306-9). The human VEGF-A gene is structured in eight exons that give rise to four main isoforms by alternative splicing (Tischer et al., J Biol Chem 266:11947-54). The isoform VEGF165, the most physiologically relevant, is secreted by both cancerous and noncancerous cells. However, a significant fraction remains bound to the cell surface and the extracellular matrix, which is mediated by its heparin-binding properties (Park et al., Mol Biol Cell 4:1317-26). It has been shown that most human tumors overexpress VEGF, which is associated with tumor progression and poor prognosis in colorectal, lung, breast, pancreatic, and gastrointestinal carcinomas and melanoma (Takahashi et al., Cancer Res 55:3964-8; Fontanini et al., J Natl Cancer Inst 89:881-6; Berns et al., Clin Cancer Res 9:1253-8; Ikeda et al., Br J Cancer 79:1553-63; Takahashi et al., Clin Cancer Res 2:1679-84; Goydos et al., Clin Cancer Res 9:5962-7). Human VEGF exerts its functions through binding to two related receptors, VEGF receptor (VEGFR) 1 (Flt-1) and VEGFR2 (Flk-1 or KDR), which are expressed mostly on endothelial cells (Ferrara et al., Nat Med 9:669-76; Neufeld et al., FASEB 13:9-22). VEGFR2 is the primary receptor for transmitting VEGF signals and is a transmembrane protein with an intracellular tyrosine kinase-active end. The induction of this tyrosine kinase by VEGF fastening initiates a cascade of phosphorylation of other signaling molecules, resulting in microvascular permeability, endothelial cell proliferation, invasion, migration, and survival (Millauer et al., Cell 72:835-46; Zeng et al., J Biol Chem 276:26969-79). Interestingly, VEGFRs are also expressed on tumor cells, including those from non-small cell lung carcinoma, leukemia, prostate carcinoma, and breast carcinoma (Decaussin et al., J Pathol 188:369-77; Bellamy et al., Cancer Res 59:728-33; Ferrer et al., Urology 54:567-72; Price et al., Cell Growth Differ 12:129-35). Although the significance of this observed expression pattern is still under investigation, it is intriguing to hypothesize that circulating VEGF could bind to its receptor on tumor cells to form VEGF-VEGFR complex, thereby stimulating tumor growth and metastasis.

Anti-VEGF monoclonal antibody (mAb; bevacizumab, Avastin) is a murine-derived recombinant mAb with a human IgG1 framework. It is capable of binding and neutralizing all biologically active isoforms of VEGF, thus potently blocking VEGF (Kim et al., Growth Factors 7:53-64; Presta et al., Cancer Res 57:4593-9). Bevacizumab was shown as having no direct effect on the proliferation of tumor cell lines. Rather, it was concluded that its target is the endothelial cells and the tumor blood supply (Kim et al., Nature 362:841-4). Thus, the proposed mechanism of action of bevacizumab is the blocking of secreted VEGF, resulting in regression to tumor microvessels, normalization of surviving mature vasculature, and inhibition of vessel growth and neovascularization (Presta et al., Cancer Res 57:4593-9; Willet et al., Nat Med 10:145-7). Although bevacizumab uses the human IgG1 framework, which itself is capable of activating complement, it has not been shown to activate complement or to be cytotoxic to tumor cells, neither in vitro nor in vivo. This tendency to attack tumor cells is a mechanism exhibited by other antitumor antibodies, such as rituximab (anti-CD20 mAb; Gelderman et al., Trends Immunol 25:158-64).

β-Glucan, a pathogen-associated molecular pattern, has shown therapeutic benefits in a variety of animal disease models. Previous in vitro studies have shown that the small molecular mass of yeast-derived β-glucan binds a lectin-like domain within the COOH-terminal region of the CD11b subunit of leukocyte complement receptor 3 (CR3; CD11b/CD18, aMh2 integrin, Mac-1; refs. Thorton et al., J Immunol 156:1235-46, Xia et al., J Immunol 162:2281-90). β-Glucans prime CR3 of neutrophils, macrophages, and natural killer cells for cytotoxicity against tumors opsonized with iC3b as a result of complement activation by antitumor mAbs or natural antibodies. Dual occupancy of leukocyte CR3 by the I-domain ligand iC3b and the lectin-like domain ligand β-glucan leads to degranulation and cytotoxic responses (Li et al., J Immunol 177:1661-9; Tsikitis et al., J Immunol 173:1284-91). Further studies have shown that successful β-glucan-mediated tumor immunotherapy requires tumor-reactive antibodies that activate complement and deposit iC3b on tumor cells and CR3 on leukocytes (Yan et al., J Immunol 163:3045-52; Hong et al., Cancer Res 63:9023-31). In addition, neutrophils have been identified as the predominate effector cells for β-glucan-mediated tumor therapy (Hong et al., Cancer Res 63:9023-31; Allendorf et al., J Immunol 174:7050-6).

The current study hypothesized that bevacizumab, in addition to its conventional effects on circulating VEGF, also binds membrane-bound VEGF on tumor cells, leading to complement activation and iC3b deposition on tumors. This effect can be augmented by coadministration of yeast-derived β-glucan, which results in the synergistic and heightened antitumor effects for tumor therapy. This study has a double therapeutic consequence. First, the need for testing membrane-bound VEGF expression in tumors was established. Second, an augmented therapeutic efficacy against tumors mediated by the combination of bevacizumab and β-glucan therapy was revealed, one that allows the clinical use of this therapy without added chemotherapy or adverse effects.

Bevacizumab (Avastin™) is a murine-derived mAb with human IgG1 framework. Its proposed antitumor mechanism of action involves blocking circulating vascular endothelial growth factor (VEGF), thus preventing its binding to the VEGF receptor on vascular endothelium (Ferrara, Endocr Rev 25:581-611). The predominant and most physiologically relevant VEGF isoform (VEGF₁₆₅) can either remain membrane-bound or be secreted (Ferrer et al., Urology 54:567-572; Bellamy et al., Cancer Res 59:728-733; Decaussin et al., J Pathol 188:369-377; Miralem et al., Oncogene 20:5511-5524; Price et al., Cell Growth Differ 12:129-135). In advanced NSCLC, bevacizumab in combination with chemotherapy has shown to increase the response rate and the time to progression (Johnson et al., J Clin Oncol 22:2184-2191). In metastatic NSCLC, bevacizumab added to carboplatin/paclitaxel, showed statistically significant improved response rate, progression-free survival and median survival (Sandler et al., N Engl J Med 355:2542-2550). Human IgG1 isotype antibodies can efficiently activate complement resulting in the deposition of iC3b and acts as an opsonin on the surface of tumor cells, thereby enhancing β-glucan mediated CR3-dependent cellular cytotoxicity (Li et al., J Immunol 177:1661-1669; Yan et al., Endocr Rev 25:581-611; Hong et al., Cancer Res 63:9023-9031; Hong et al., J Immunol 173:797-806; Allendorf et al., J Immunol 174:7050-7056). Therefore, the IgG1 antibody bevacizumab is capable of binding surface-bound VEGF on NSCLC tumor cells, thereby activating complement and synergizing with β-glucan to elicit CR3-dependent cellular cytotoxicity.

VEGF is an endothelial cell-specific mitogen and a major regulator for angiogenesis. VEGF is overexpressed in most human tumors (Ferrara, Endocr Rev 25:581-611) and is crucial to tumor growth. It stimulates abundant angiogenesis that allows the tumor to grow exponentially as well as provides the hematogenous route for metastasis. Thus, the proposed mechanism of action for bevacizumab is by blocking secreted VEGF resulting in regression of tumor microvessels, normalization of surviving mature vasculature, and inhibition of vessel growth and neovasculatization. Bevacizumab has been shown to have no effect on proliferation of tumor cell lines and this has been used to conclude that its target is not the tumor cells but the endothelial cells and the tumor blood supply (Kim et al., Nature 362:841-844). Although bevacizumab uses human IgG1 framework, it has not been shown to activate complement or to be cytotoxic to tumor cells, neither in vivo nor in vitro.

CR3 is widely expressed on the surface of all phagocytes including neutrophils, eosinophils, and basophils as well as on the surface of monocytes, macrophages, and NK cells (Ross, Crit. Rev Immunol 20:197-222; Klein et al., Mol Immunol 27:1343-1347; Ross et al., Clin Exp Immunol 92:181-184). It has been shown that neutrophil CR3-dependent phagocytosis or degranulation in response to iC3b-opsonized yeast required ligation of two distinct binding sites in CR3, one for iC3b and a second site for β-glucan. Subsequent research mapped each of these binding sites to domains within the α-chain of CR3, CD11b. Furthermore, the lectin site was mapped to a region of CD11b located C-terminal to the I-domain (Thornton et al., J Immunol 156:1235-1246; Xia et al., J Immunol 162:7285-7293). Induction of CR3-depedent cell-mediated cytotoxicity (CR3-DCC) requires dual ligation of CR3 to both iC3b and yeast β-glucan (Vetvicka et al., J Clin Invest 98:50-61; Xia et al., J Immunol 162:2281-2290). C3-opsonized yeast presents iC3b in combination with β-glucan, such that both of these domains of CR3 become attached to the yeast, stimulating phagocytosis and cytotoxic degranulation. In contrast to microorganisms, tumor cells lack β-glucan. The lack of similar CR3-binding β-glucan on human cells explains the inability of CR3 to mediate phagocytosis or cytotoxicity of tumor cells opsonized with iC3b. iC3b-opsonized tumor cells mediated by naturally occurring anti-tumor Abs or anti-tumor mAbs engage only the I-domain of CD11b and not the lectin site. Soluble β(1,3) glucan polysaccharides isolated from fungi can bind to the lectin site of CR3 with high affinity and prime the receptor for subsequent cytotoxic activation by iC3b-opsonized tumor cells that are otherwise inert in stimulating CR3-DCC. Therapy failure in C3- or CR3-deficient mice (C3^{-/-}, CR3^{-/-}) indicates the requirement for both iC3b deposited on tumor cells mediated by complement-activating mAbs or naturally occurring Abs and its receptor CR3 on phagocytes (Hong et al., Cancer Res 63:9023-9031; Hong et al., J Immunol 173:797-806).

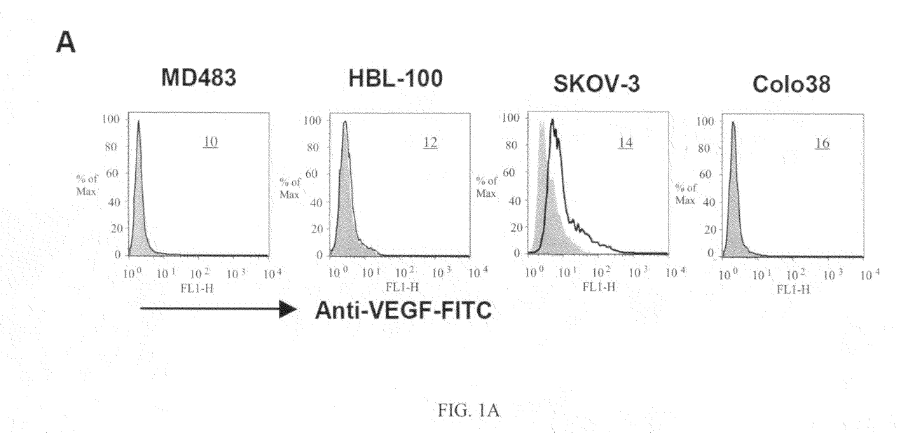

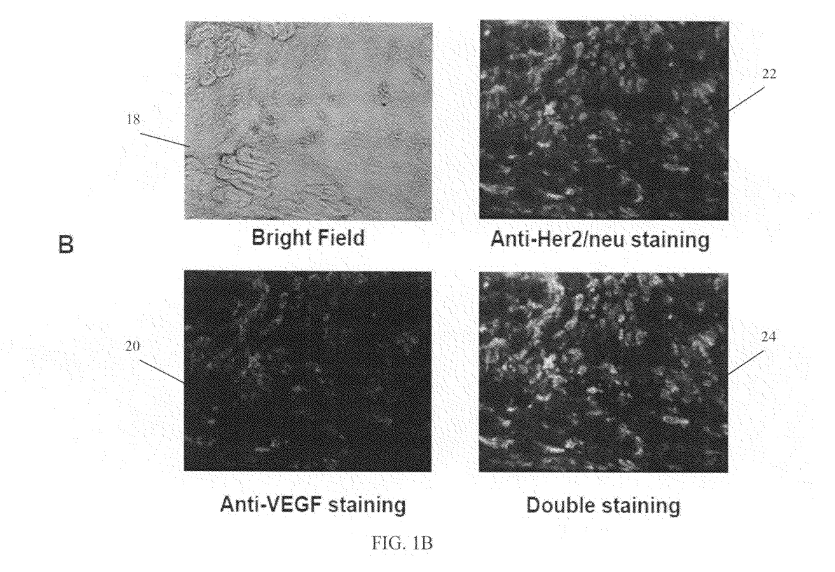

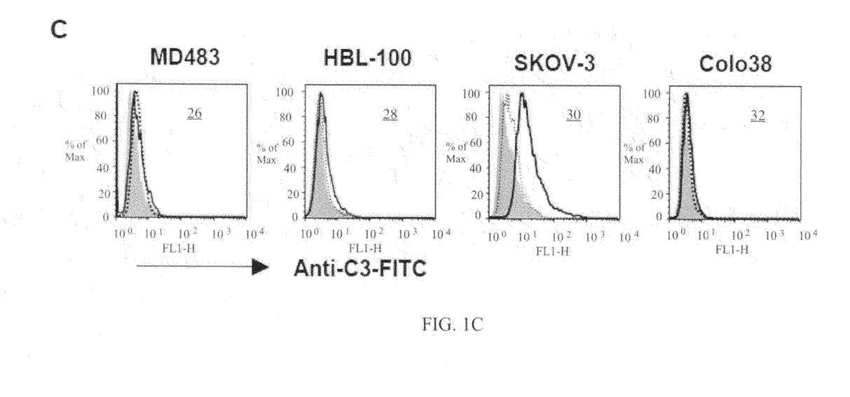

The anti-VEGF mAb bevacizumab does not have a major cytotoxic antitumor action and although most tumors are known to be VEGF producers, little is known about the significance of the expression of membrane-bound VEGF on tumor cells. Detection of membrane-bound VEGF expression on tumor cell lines was initially carried out with human carcinoma cell lines. Cell lines were cultured in DMEM with 10% newborn calf serum, MEM non-essential amino acids, 100 units/mL penicillin, 100 μg/mL streptomycin and 2 mmol/L L-glutamine. Tumor cells were harvested and Fc receptors were blocked by incubation with anti-CD32/CD16 mAb. Cells were then stained with labeled anti-VEGF mAb or isotype control and analyzed by flow cytometry. FIG. 1A shows the results of human breast carcinomas MDA-MB-483, histogram 10, and HBL-100, histogram 12; human ovarian carcinoma SKOV-3, histogram 14, and human melanoma Colo38, histogram 16, cells stained with fluorescein-labeled anti-VEGF mAb (bold line) or fluorescein-labeled isotype (filled gray) and assessed by flow cytometry. As the results indicate, only SKOV-3 tumors express high levels of membrane-bound VEGF.

To further confirm indeed SKOV-3 tumors express membrane-bound VEGF in vivo, human ovarian carcinoma SKOV-3 cells were implanted into SCID mice. Because SKOV-3 cells express high levels of Her-2/neu onco-protein, anti-Her-2 mAb was used to track tumor cells. After tumors reached 7-8 mm in diameter, mice were sacrificed and tumors were removed and snap-frozen. Tumor sections were stained with anti-VEGF-PE mAb or anti-Her-2/neu-FITC or both. Solid tumors were excised and snap frozen in tissue freezing medium. Tumor sections were first blocked with 3% bovine serum albumin/PBS and then stained with the antibodies. Results are shown in FIG. 1B. Panel 18 shows a field of SKOV-3 cells using bright field microscopy. Panel 20 shows anti-VEGF staining, panel 22 shows anti-Her-2/neu staining and panel 24 shows the overlay of both panels 20 and 22. As indicated, SKOV-3 tumors exhibited high-density expression of Her-2/neu that co-localized with anti-VEGF mAb staining. Thus, SKOV-3 tumors indeed express membrane-bound VEGF.

Next, the ability of anti-VEGF mAb to bind surface-bound VEGF and activate complement, which leads to iC3b deposition, was examined. SKOV-3 cells were mixed with anti-VEGF mAb in the presence of a 1:4 dilution of mouse serum (complement source). For every one million tumor cells, a 100 μL volume of diluted mouse serum containing 10 μg/mL working dilution of anti-VEGF mAb was used. Tumor cells were mixed and incubated at 37° C. for 30 min. The cells were washed in ice cold flow cytometry staining buffer and the cell pellet was resuspended in 100 mL diluted detecting antibody. The cells were incubated on ice for 30 min. and washed twice. Propidium iodide was used to exclude dead cells. The results of cell lines MDA-MB-483, HBL-100, SKOV-3 and Colo38 are shown in histograms 26, 28, 30 and 32 of FIG. 1C, respectively. Cells incubated with anti-VEGF mAb in the presence of complement and stained with anti-mouse iC3b-FITC mAb are represented by bold lines. Controls included cells incubated with mouse complement (dotted line) or without mouse complement (filled gray) and then stained with anti-mouse iC3b-FITC mAb. Anti-VEGF mAb could bind membrane-bound VEGF on SKOV-3 cells and efficiently activate complement. All other tumor cell lines negative for membrane-bound VEGF expression were also negative for complement activation.

As described above, β-glucan functions with anti-tumor antibodies to activate complement receptor 3 (CR3) and recruit neutrophils that mediate CR3 (iC3b-receptor)-dependent cytotoxicity of tumors coated with iC3b. Anti-VEGF mAb bevacizumab is an IgG1 isotype antibody that binds membrane-bound VEGF and efficiently activates complement as indicated by FIG. 1C. To test the possible synergy of β-glucan and bevacizumab in vitro, a CR3-dependent cytotoxic assay was performed. SKOV-3 cells were incubated with anti-VEGF mAb or anti-Her-2/neu mAb in the presence of human complement. Cells were then washed and co-cultured with human neutrophils (E:T ratio=20:1) in the presence of soluble β-glucan. Cytotoxicity was calculated as in Li et al., J Immunol 177:1661-1669. The cytotoxicity mediated by bevacizumab alone was 0.2% and bevacizumab plus β-glucan (A) resulted in 19% cytotoxicity. Anti-Her-2/neu mAb (trastuzumab) (B) was used as a positive control since SKOV-3 cells overexpress HER2/neu. It also synergized with β-glucan to elicit CR3-dependent cellular cytotoxicity (CR3-DCC).

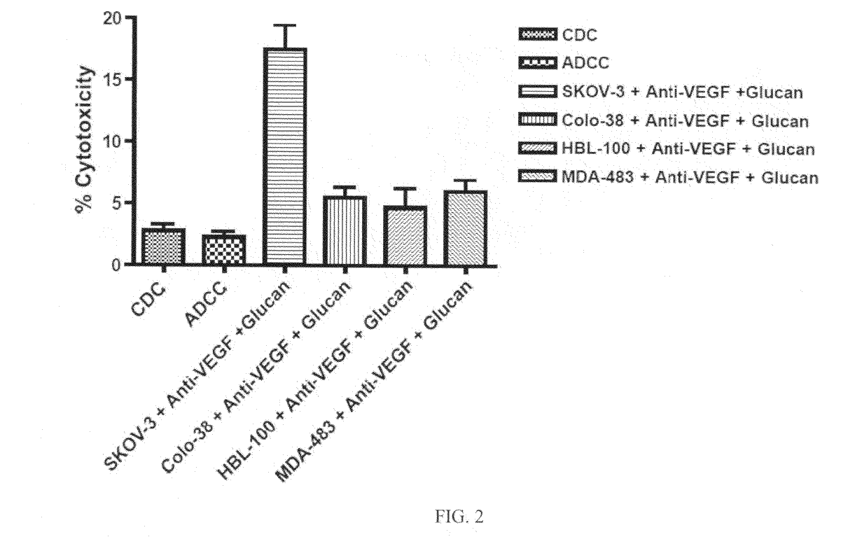

For additional evidence, tumor cells were incubated with anti-VEGF mAb in the presence of human complement to achieve iC3b opsinization on tumor cells and then cocultured with neutrophils either primed with β-glucan or unprimed. As shown in FIG. 2, the complement-dependent cytotoxicity (SKOV-3 cells with anti-VEGF mAb in the presence of mouse complement) mediated by bevacizumab was about 3%. Antibody-dependent cellular cytotoxicity (SKOV-3 cells with anti-VEGF mAb plus mouse complement plus neutrophils without β-glucan) was similar to complement-dependent cytotoxicity. This data reaffirms that bevacizumab's mechanism of action is independent of immune effector functions including complement-dependent cytotoxicity and antibody-dependent cytotoxicity. Strikingly, bevacizumab plus β-glucan resulted in about 18% cytotoxicity, and again, iC3b-opsonized SKOV-3 tumor cells but not the other tumor cells, which do not express membrane-bound VEGF (FIG. 2).

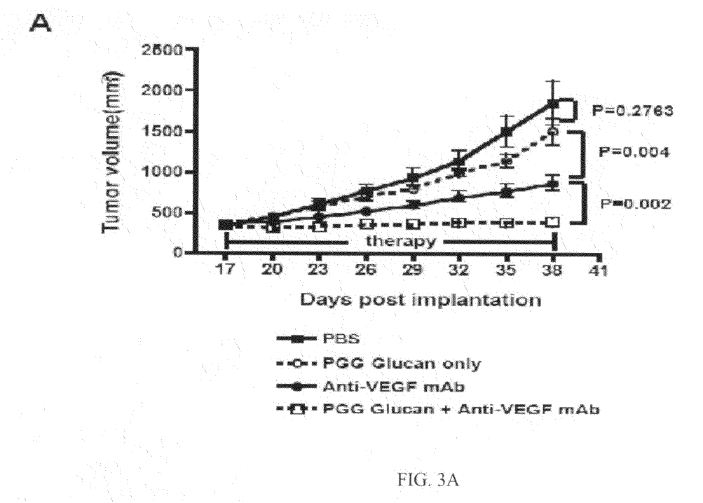

To determine if the combination of soluble β-glucan and bevacizumab has an augmented anti-tumor effect in vivo compared to therapy with bevacizumab alone, 36 ICR SCID mouse xenografts with SKOV-3 cells were produced. After SKOV-3 cell s.c. implantation, tumors were allowed to grow for over 17 days until they were 300 mm³. The mice were then assigned to four different treatment groups: PBS treated as control (▪), 1.2 mg β-glucan treated twice weekly (▾), 0.2 mg bevacizumab treated every third day (▴) and β-glucan+bevacizumab (♦), given intravenously for 4 weeks. Mice were sacrificed when the tumors reached 15 mm in diameter. As shown in FIG. 3A, tumors in the PBS-treated mice reached 1800±800 mm³; tumors in the β-glucan-treated group reached 1500±500 mm³ (P=0.3 when compared to control group); tumors in the bevacizumab-treated group reached 800±250 mm³ (P=0.004 when compare to β-glucan); and tumors in the β-glucan+bevacizumab-treated group reached 400±150 mm³ (P=0.002 when compared to bevacizumab alone). These data suggest that anti-VEGF mAb exhibited a significant tumor regression as previously reported. Anti-VEGF mAb in conjunction with β-glucan therapy achieved drastically therapeutic efficacy as compared to any of the other groups. Most of these tumors still retained the sizes before therapy. More importantly, as shown in FIG. 3B, mice treated with β-glucan plus anti-VEGF mAb had statistically significant higher survival at 100 days than those treated with anti-VEGF mAb alone or any of the other groups.

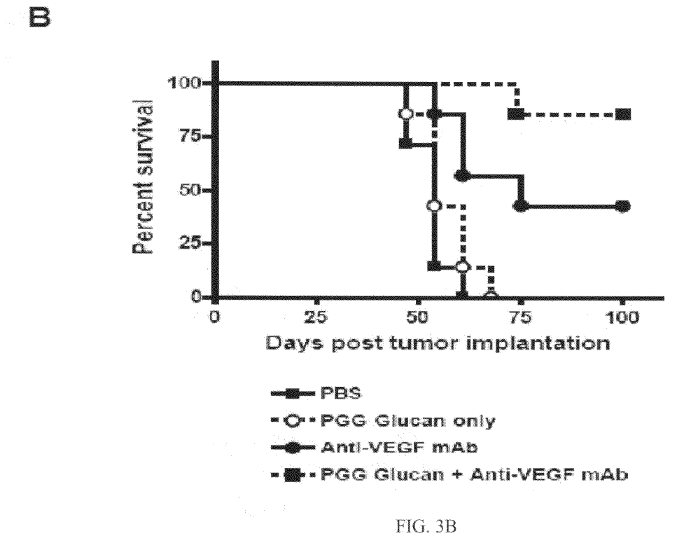

Additional evidence was gathered to show efficacy of in vivo therapy. ICR SCID mice implanted with SKOV-3 cells were compared to ICR SCID mice implanted with Colo38 cells as a control. As shown in FIG. 4A, in the SKOV-3 xenograft model, mice receiving β-glucan only did not show any significant tumor regression compared with untreated animals (P=0.14). Mice treated with anti-VEGF mAb alone exhibited a significantly reduced tumor burden compared with untreated or treated with β-glucan only (P<0.0001 with respect to β-glucan only-treated animal). But strikingly, mice receiving β-glucan in combination with anti-VEGF mAb therapy had significantly smaller tumors compared with anti-VEGF mAb treatment alone (P<0.05). Most of these tumors still retained their sizes before therapy. More importantly, 86% of these mice achieved long-term survival compared with 43% of tumor-bearing mice treated with anti-VEGF mAb only (FIG. 4B). In the Colo38 xenograft model, mice treated with anti-VEGF mAb alone also showed a significantly reduced tumor burden compared with untreated or treated with β-glucan only (P<0.01). However, no significant difference was observed in tumor-bearing mice treated with anti-VEGF mAb alone or anti-VEGF mAb in combination with β-glucan (P=0.67; FIG. 4C). These data suggest that the addition of β-glucan to anti-VEGF mAb therapy significantly enhances the regression of the membrane-bound VEGF-positive SKOV-3 tumors and long-term survival.

Interaction between the complement activation product iC3b and the I-domain of CR3 provides the first signal for CR3 priming. Engagement of β-glucan with the lectin-like domain of CR3 is additionally required for the full activation of CR3. The dual ligation of CR3 leads to neutrophil CR3-dependent cellular cytotoxicity (Li et al., J Immunol 177:1661-1669). Neutrophil trafficking within tumors is also critical for successful combined β-glucan with anti-tumor mAb therapy (Allendorf et al., J Immunol 174:7050-7056). Therefore, immunohistochemistry studies were carried out to examine complement activation and neutrophil infiltration within tumors after treatment with different regimens.

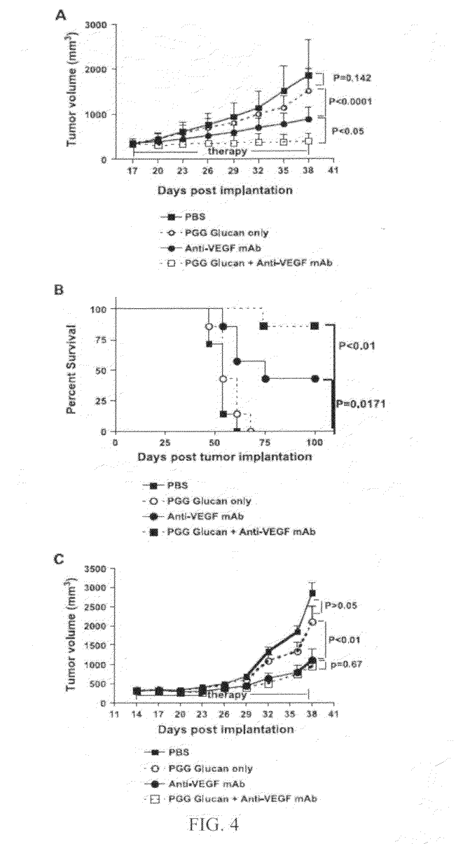

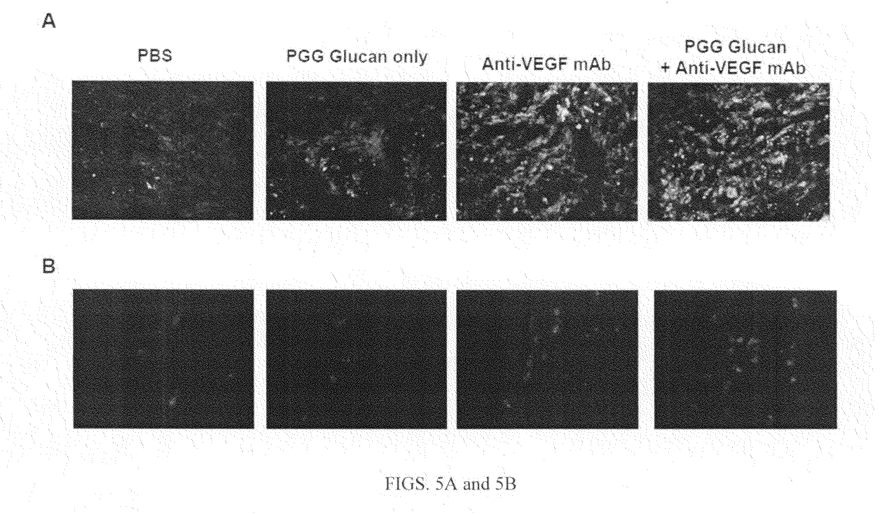

First, the tumor sections from four groups were stained with an anti-iC3b-FITC and anti-VEGF-PE mAbs. As shown in FIG. 5A, massive iC3b deposition occurred in tumors treated with anti-VEGF mAb with or without β-glucan, indicating that indeed anti-VEGF mAb is capable of activating complement in vivo. While there was no iC3b deposition on the tumors from mice treated with β-glucan alone or PBS. Second, the tumor sections were stained with anti-Gr-1-PE mAb to detect neutrophil infiltration. As indicated in FIG. 5B, the tumors from mice receiving β-glucan plus anti-VEGF mAb (bevacizumab) therapy had a significantly increased number of infiltrating neutrophils than the mice in the PBS or β-glucan alone treated groups. Interestingly, tumors treated with anti-VEGF mAb alone also had significant neutrophil infiltration, suggesting that anti-VEGF mAb has a unique function in which the tumor microenvironment is altered such that it subsequent inflammatory neutrophil infiltration occurs within tumors.

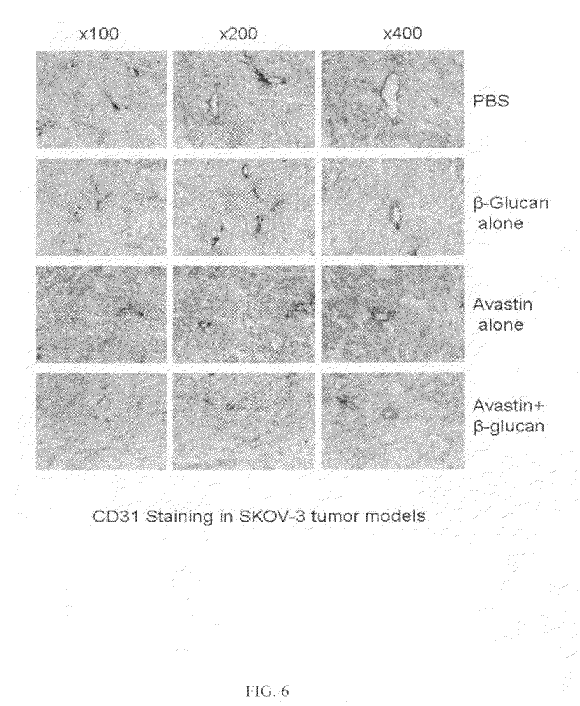

To observe tumor blood vessel development after anti-VEGF mAb therapy, the tumor sections were stained with anti-CD31-biotin mAb. As shown in FIG. 6, decreased tumor microvessel density was observed in the tumors from mice treated with a combination of β-glucan plus anti-VEGF mAb (bevacizumab/Avastin) or anti-VEGF mAb alone when compared to the groups treated with either PBS or β-glucan alone. This further suggests that the mechanism of action of anti-VEGF mAb is to block angiogenesis.

Here human ovarian tumor cells were shown to express membrane-bound VEGF both in vitro and in vivo. This unique expression pattern leads to the complement activation and iC3b opsonization on tumor cells mediated by anti-VEGF mAb, bevacizumab. More importantly, the augmented therapeutic efficacy is achieved by combined PGG β-glucan with anti-VEGF mAb therapy. The enhanced anti-tumor responses and long-term survival for tumor-bearing mice treated with the combined therapy seem to be associated with intratumor massive complement activation and neutrophil infiltration. Therefore, this strategy may offer its clinical benefits for tumor patients with membrane-bound VEGF expression.

Bevacizumab in combination with chemotherapy has been approved by the Food and Drug Administration for use in colorectal and lung cancer treatments (Hicklin et al., J Clin Oncol 23:1011-27). Although the mechanism of action of bevacizumab has not been fully elucidated, the proposed mechanism of action is via blockade of circulating VEGF secreted by cancer cells or cancer stromal cells and is independent of immune effector mechanisms, such as complement-dependent cytotoxicity or antibody-dependent cellular cytotoxicity. The in vitro cytotoxicity experiments discussed above support this notion. However, some tumors not only secrete soluble VEGF into the extracellular matrix but also express a membrane-bound form of VEGF. This study showed that human ovarian carcinoma SKOV-3 expresses membrane-bound VEGF both in vitro and in vivo. Additionally, VEGFRs are also expressed on tumor cells (Decaussin et al., J Pathol 188:369-77; Bellamy et al., Cancer Res 59:728-33; Ferrer et al., Urology 54:567-72; Price et al., Cell Growth Differ 12:129-35). It is possible that the secreted VEGF may bind to its receptor on tumor cells forming a ligand-receptor complex. Indeed, studies have shown that functional VEGF/VEGFR2 autocrine loops are present in human leukemia and support leukemic cell survival and migration (Dias et al., Proc Natl Acad Sci USA 98:10857-62; Dias et al., J Clin Invest 106:511-21). Therefore, anti-VEGF mAb may bind surface-bound VEGF or VEGF-VEGFR complex and initiate potential immunologic consequences. Although anti-VEGF mAb bevacizumab itself does not have significant direct cytotoxicity against tumor cells, in vitro study showed that yeast-derived β-glucan is able to synergize with anti-VEGF mAb to elicit leukocyte-mediated CR3-dependent cellular cytotoxicity. These data suggest that the anti-VEGF mAb bevacizumab can be manipulated in such a way as to elicit effective antitumor immune responses. In fact, this study showed that PGG β-glucan in addition to anti-VEGF mAb therapy achieved a significantly smaller tumor burden and long-term survival with respect to anti-VEGF mAb-treated only animals. However, this synergy did not occur in Colo38 tumors, which do not express membrane-bound VEGF. The augmented therapeutic efficacy offers potential clinical benefits for cancer patients who are subject to anti-VEGF mAb therapy. This may also pose a question of whether membrane-bound VEGF expression should be included in the clinical pathologic report. Although anti-VEGF mAb therapy has been approved for clinical use, the response rate mediated by anti-VEGF mAb as an autonomous monotherapy is limited. In clinical practice, anti-VEGF mAb has been used in combination with chemotherapy, which significantly increases the therapeutic efficacy (Sandler et al., New Engl J Med 355:2542-50; Johnson et al., J Clin Oncol 22:2184-91; Ellis L M, Nat Rev Drug Discov Suppl:S8-9). However, those combination therapies also have more severe adverse effects, which limit general use for most patients. Yeast-derived PGG β-glucan is a polysaccharide and has a minimal toxicity (Yan et al., Expert Opin Biol Ther 5:691-702; Zimmerman et al., J Biol Chem 273:22014-20). The in vitro and in vivo data clearly suggest that β-glucan can significantly augment the therapeutic efficacy mediated by anti-VEGF mAb bevacizumab in membrane-bound VEGF-positive SKOV-3 tumors but not in membrane-bound VEGF-negative Colo38 tumors. The premise for this combination therapy requires tumors expressing membrane-bound VEGF. Therefore, it seems necessary to detect membrane-bound VEGF expression for patients who potentially undergo this combination therapy. Nevertheless, the combined β-glucan with anti-VEGF mAb therapy offers an alternative strategy for cancer therapy and suggests that bevacizumab therapy can be incorporated with other immune effector functions, such as β-glucan-mediated CR3-dependent cellular cytotoxicity.

Previous studies in murine syngeneic tumor models have shown that the successful β-glucan-mediated tumor therapy requires glucan-primed neutrophils traffic into tumors and iC3b opsonization on tumor cells (Hong et al., Cancer Res 63:9023-31; Allendorf et al., J Immunol 174:7050-6). The dual ligation of leukocyte CR3 leads to degranulation and cytotoxic responses to iC3b-coated tumor cells (Li et al., J Immunol 177:1661-9). This seems to be the case in the current study. Massive complement activation and intratumor neutrophil infiltration occurred in tumors treated with anti-VEGF mAb with or without β-glucan. This process is independent of β-glucan because tumor-bearing mice treated with β-glucan alone also showed the paucity of iC3b deposition and neutrophil infiltration within the tumors similar to mice treated with PBS injection. This may suggest that SKOV-3 tumors have established an immune-suppressive mechanism to escape immune surveillance. Interestingly, tumors treated with anti-VEGF mAb alone exhibited massive iC3b deposition and neutrophil infiltration.

It was previously shown that SKOV-3 tumors express high levels of membrane complement regulatory proteins, such as CD46, CD55, and CD59 (Li et al., Cancer Research 67:7421-30). Particularly, up-regulation of CD55 (decay-accelerating factor) on SKOV-3 cells protected SKOV-3 cells from complement-mediated lysis (Bjorge et al., Int J Cancer 70:14-25). Although SKOV-3 tumors express a high density of Her-2/neu oncoprotein, SKOV-3 tumors were resistant to the anti-Her-2/neu mAb treatment, even in conjunction with β-glucan when a few neutrophils were infiltrated within the SKOV-3 tumors. However, anti-VEGF mAb treatment alone led to the alteration of such a suppressive microenvironment. This seems to be independent of complement activation because both mAbs have human IgG1 frameworks and potently activate complement. It is possible that anti-VEGF mAb uses its unique antiangiogenic activity, which disrupts tumor blood vessel supply and therefore modulates the tumor microenvironment to favor immunotherapy. A recent study has shown that VEGF secreted by carcinoma cells either directly or indirectly participates in maintaining an inflammatory microenvironment (Salnikov et al., Int J Cancer 119:2795-802). Bevacizumab reduced the density of macrophages, MHC class II antigen expression by macrophages, and interleukin-1β mRNA expression. Interestingly, VEGF also stimulates CD55 production on tumor cells (Mason et al., J Biol Chem 279:41611-8). Therefore, anti-VEGF mAb bevacizumab treatment could down-regulate CD55 expression, thereby leading to potent neutrophil chemoattractant C5a release within the tumor.

This study is the first to prove that anti-VEGF mAb bevacizumab can be used in concert with other immune effector functions when it is coadministered with yeast-derived β-glucan. This strategy provides a novel mechanism of action of bevacizumab and has a significant clinical implication for combination therapies. The combination of bevacizumab and β-glucan has great potential to become part of the armament against cancer and deserves further investigation with the goal of translation into clinical practice.

The invention encompasses a composition including both a β-glucan and an VEGF antagonist. Because of the proven ability of β-glucans to treat immune dysfunction including infections and other immune problems associated with chemotherapy, radiation treatment and other cancer treatments, and VEGF antagonists have been shown to be effective in the treatment of cancer, the two active ingredients will work additively or synergistically in the treatment of proliferative disorders or immune dysfunction. Further, other pharmaceuticals are used with the composition of the invention. For example, other anti-cancer drugs are combined with a β-glucan and an VEGF antagonist for the treatment of a proliferative disorder. Also, more than one β-glucan or VEGF antagonist are used in the same composition. For example, a triple helical β-glucan is combined with a β-glucan with an aggregate number of 7 (meaning that the aggregate contains 7 β-glucan chains) which is further combined with bevacizumab.

The β-glucans used in the invention include particulate β-glucan, PGG (poly-(1-6)-β-D-glucopyranosyl-(1-3)-β-D-glucopyranose), neutral soluble β-glucan, triple helical β-glucan, IMPRIME PGG™, and β-glucans of various aggregate numbers. The above-mentioned species of β-glucans are administered separately or in various combinations. VEGF antagonists used in the composition of the invention include polyclonal and monoclonal antibodies, recombinant human/mouse chimeric monoclonal antibody, antibody fragments, other proteins and small molecules that bind specifically to the extracellular domain of the human VEGF. The above-mentioned species of VEGF receptor antagonists are administered separately or in various combinations.

The invention also encompasses a method of treating a proliferative disorder in a mammal by administering to the mammal a composition including both a β-glucan and a VEGF antagonist. The method may further comprise the administration of other anti-cancer drugs with the β-glucan and a VEGF antagonist. The invention also encompasses a method of treating an immune dysfunction in a mammal by administering to the mammal a composition including both a β-glucan and a VEGF antagonist.

The compositions may, if desired, be presented in a pack or dispenser device and/or a kit, which may contain one or more unit dosage forms containing the active ingredients. The pack may for example comprise metal or plastic foil, such as a blister pack. The pack or dispenser device may be accompanied by instructions for administration.

β-Glucans

The β-glucan preparations of this invention are insoluble glucan particles or prepared from insoluble glucan particles. Manners et al., Biol. J., 135:19-30, (1973). β-glucan is also referred to herein as PGG (poly-(1-6)-β-D-glucopyranosyl-(1-3)-β-D-glucopyranose). A β-glucan polysaccharide can exist in at least four distinct conformations: single disordered chains, single helix, single triple helix and triple helix aggregates. The terms “neutral soluble β-glucan” and “neutral soluble glucan” are intended to mean an aqueous soluble β-glucan having a unique triple helical conformation that results from the denaturation and re-annealing of aqueous soluble glucan. Single chains are also isolated and used, i.e., not substantially interacting with another chain. Three single helix chains can combine to form a triple helix structure which is held together by interchain hydrogen bonding. Two or more β-glucan triple helices can join together to form a triple helix aggregate. Preparations of the β-glucan can comprise one or more of these forms, depending upon such conditions as pH and temperature.

Glucan particles which are particularly useful as starting materials in the present invention are whole glucan particles described by Jamas et al., in U.S. Pat. Nos. 4,810,646, 4,992,540, 5,082,936 and 5,028,703, the teaching of all are hereby incorporated herein by reference. The source of the whole glucan particles can be the broad spectrum of glucan-containing fungal organisms which contain β-glucans in their cell walls. Whole glucan particles obtained from the strain Saccharomyces cerevisiae R4 (NRRL Y-15903; deposit made in connection with U.S. Pat. No. 4,810,646) and R4Ad (ATCC No. 74181) are particularly useful. The structurally modified glucans hereinafter referred to as “modified glucans” derived from S. cerevisiae R4 are potent immune system activators, as described in Jamas et al. in U.S. Pat. No. 5,504,079, the teachings of which are hereby incorporated herein by reference.

The whole glucan particles utilized in this present invention can be in the form of a dried powder, as described by Jamas et al., in U.S. Pat. Nos. 4,810,646, 4,992,540, 5,082,936 and 5,028,703. For the purpose of this present invention it is not necessary to conduct the final organic extraction and wash steps described by Jamas et al.

The soluble glucans are branched polymers of glucose containing β(1-3) and β(1-6) linkages in varying ratios depending on the organism and processing conditions employed. The glucan preparations contain neutral glucans, which have not been modified by substitution with functional (e.g., charged) groups or other covalent attachments. The biological activity of PGG glucan can be controlled by varying the average molecular weight and the ratio of β(1-6) to β(1-3) linkages of the glucan molecules, as described by Jamas et al. in U.S. Pat. Nos. 4,810,646, 4,992,540, 5,082,936 and 5,028,703. The average molecular weight of soluble glucans produced by the present method is generally from about 10,000 to about 500,000 daltons, preferably from about 30,000 to about 50,000.

Neutral Soluble β-glucan

Neutral soluble β-glucan (also referred to as IMPRIME PGG™) has been shown to increase the number of neutrophils and monocytes as well as their direct infection fighting activity (phagocytosis and microbial killing). However, the neutral soluble β-glucan does not stimulate the production of biochemical mediators, such as IL-1, TNF and leukotrienes, that can cause detrimental side effects such as high fever, inflammation, wasting disease and organ failure. These advantageous properties make neutral soluble glucan preparations useful in the prevention and treatment of infection because they selectively activate only those components of the immune system responsible for the initial response to infection, without stimulating the release of certain biochemical mediators that can cause adverse side effects. The solution containing the neutral soluble β-glucan also lacks the toxicity common to many immunomodulators.

The neutral soluble β-glucans of this invention are composed of glucose monomers organized as a β(1-3) linked glucopyranose backbone with periodic branching via β(1-6) glycosidic linkages. The neutral soluble glucan preparations contain glucans, which have not been substantially modified by substitution with functional (e.g., charged) groups or other covalent attachments. The general structure of the neutral soluble glucan is shown in FIG. 1 of U.S. Pat. No. 5,488,040, incorporated herein, by reference. One biologically active preparation of this invention is a conformationally purified form of β-glucan produced by dissociating the native glucan conformations and re-annealing and purifying the resulting unique triple helical conformation. The unique conformation of the neutral soluble glucan contributes to the glucan's ability to selectively activate the immune system without stimulating the production of detrimental biochemical mediators. One method of making soluble β-glucans are described in U.S. patent application Ser. Nos. 11/818,741 and 11/818,804, which are incorporated by reference.

The soluble glucan preparations of this invention are prepared from insoluble glucan particles, preferably derived from yeast organisms as described herein. Other strains of yeast that can be used include Saccharomyces delbrueckii, Saccharomyces rosei, Saccharomyces microellipsodes, Saccharomyces carlsbergensis, Schizosaccharomyces pombe, Kluyveromyces lactis, Kluyveromyces fragilis, Kluyveromyces polysporus, Candida albicans, Candida cloacae, Candida tropicalis, Candida utilis, Hansenula wingeri, Hansenula arni, Hansenula henricii, Hansenula americana. A procedure for extraction of whole glucan particles is also described herein.

β-glucan in an Aggregate Conformation

The “aggregate number” of a β-glucan conformation is the number of single chains which are joined together in that conformation. The aggregate number of a single helix is 1, the aggregate number of a single triple helix is 3, and the aggregate number of a triple helix aggregate is greater than 3. For example, a triple helix aggregate consisting of two triple helices joined together has an aggregate number of 6.

The aggregate number of a β-glucan sample under a specified set of conditions can be determined by determining the average molecular weight of the polymer under those conditions. The β-glucan is then denatured, that is, subjected to conditions which separate any aggregates into their component single polymer chains. The average molecular weight of the denatured polymer is then determined. The ratio of the molecular weights of the aggregated and denatured forms of the polymer is the aggregate number. A typical β-glucan composition includes molecules having a range of chain lengths, conformations and molecular weights. Thus, the measured aggregate number of a β-glucan composition is the mass average aggregate number across the entire range of β-glucan molecules within the composition. It is to be understood that any reference herein to the aggregate number of a β-glucan composition refers to the mass average aggregate number of the composition under the specified conditions. The aggregate number of a composition indicates which conformation is predominant within the composition. For example, a measured aggregate number of about 6 or more is characteristic of a composition in which the β-glucan is substantially in the triple helix aggregate conformation.

The present invention also provides a soluble β-glucan composition, which comprises a triple helix aggregate conformation under physiological conditions.

In another embodiment, the present invention provides a method of preparing a soluble β-glucan composition having an aggregate number greater than that of a starting soluble β-glucan composition. The method comprises separating a high molecular weight portion from a starting soluble β-glucan composition. The high molecular weight portion is enriched in the triple helix aggregate conformation compared to the starting composition. The starting composition can be, for example, a β-glucan composition having an aggregate number less than about 6 under specified conditions. In one embodiment, the high molecular weight fraction, which is separated from the starting composition, is substantially in a triple helix aggregate conformation under physiological conditions. The high molecular weight portion can be any portion of the starting composition, as long as it has a greater average molecular weight than that of the starting composition. In one embodiment, the isolated portion represents about 60% or less, by weight, of the starting composition. The fraction of the starting composition isolated will depend upon the dispersion of molecular weights within the starting composition and the aggregate number desired and can be readily determined by one of skill in the art.

The high molecular weight portion can be separated from the starting composition using a variety of techniques. In a preferred embodiment, the high molecular weight portion is separated from the remainder of the starting composition using gel permeation chromatography (GPC). In this embodiment, the high molecular weight portion is separated from the starting composition by a method comprising the steps of (1) directing a β-glucan composition through a gel permeation chromatography column, and (2) collecting a high molecular weight fraction or a high molecular weight portion of a fraction of the starting composition.

In one embodiment, the starting β-glucan composition is separated into two or more fractions by GPC. In this case, the faster eluting fraction is a high molecular weight portion of the starting composition and all or a part of this fraction can be collected. In another embodiment, the starting β-glucan composition elutes as a single fraction or two or more overlapping fractions. In this case, the leading edge of the fraction or overlapping fractions can be collected.

The “leading edge” of a fraction eluting from a chromatography column is the portion of the fraction which elutes first. For example, if the fraction elutes in a given volume of eluent, the first 10 to 50% by volume of the fraction can be collected. The amount of the β-glucan fraction to be collected depends upon the nature of the original β-glucan composition, for example, the distribution of molecular weights and conformations, and the chromatography conditions, such as the type of GPC column employed, the eluent and the flow rate. Optimization of these parameters is within the ordinary level of skill in the art. β-Glucan molecules having higher aggregate numbers are expected to elute first. Therefore, if the portion collected has an aggregate number under physiological conditions which is lower than desired, the original β-glucan composition can be fractionated again, and a smaller leading edge portion can be collected to obtain a β-glucan composition having a larger aggregate number under physiological conditions. Preferably, the parameters are optimized using an analytical scale GPC column.

A suitable β-glucan composition having an aggregate number at physiological temperature of less than about 6 is a glucan composition previously described in U.S. Pat. No. 5,622,939. Preparative scale GPC can be performed to fractionate such a composition. For example, if the β-glucan composition elutes from the GPC column as a single band, the earlier-eluting, or leading edge, portion of the elution band can be collected to yield a glucan composition having an aggregate number greater than about 6. Such a β-glucan composition will have an increased triple helix aggregate conformer population at physiological temperature and pH compared to the original preparation.

The present invention also provides a method of preparing a soluble β-glucan composition having an aggregate number lower than that of a starting soluble β-glucan composition. The method comprises separating a low molecular weight portion from a starting soluble β-glucan composition. The low molecular weight portion is enriched in a single triple helix and/or single helix conformation compared to the starting composition. In one embodiment, the low molecular weight portion, which is separated from the starting composition is substantially in a single triple helix conformation under physiological conditions. The low molecular weight portion can be any portion of the starting composition, as long as it has a lower average molecular weight than that of the starting composition. In one embodiment, the isolated portion represents about 60% or less, by weight, of the starting composition. The fraction of the starting composition separated will depend upon the dispersion of molecular weights within the starting composition and the aggregate number desired and can be readily determined by one of skill in the art.

The low molecular weight portion can be separated from the starting composition using a variety of techniques. In a preferred embodiment, the low molecular weight portion is separated from the remainder of the starting composition using gel permeation chromatography. In this embodiment, the high molecular weight portion is separated from the starting composition by a method comprising the steps of (1) directing a glucan composition through a gel permeation chromatography column, and (2) collecting a low molecular weight fraction or a low molecular weight portion of a fraction of the starting composition.

In one embodiment, the starting β-glucan composition is separated into two or more fractions by GPC. In this case, the more slowly eluting fraction is a low molecular weight portion of the starting composition and all or a part of this fraction can be collected. In another embodiment, the starting β-glucan composition elutes as a single fraction or two or more overlapping fractions. In this case, the trailing edge of the fraction or overlapping fractions can be collected.

The “trailing edge” of a fraction eluted from a chromatography column is that portion of the fraction which elutes last. For example, if the fraction elutes in a given volume of eluent, the last 10 to 50% of the fraction can be collected. The amount of the β-glucan fraction to be collected depends upon the nature of the original β-glucan composition, for example, the distribution of molecular weights and conformations, and the chromatography conditions, such as the type of gel permeation chromatography column employed, the eluent and the flow rate. Optimization of these parameters is within the ordinary level of skill in the art. β-Glucan molecules which adopt a single triple helix conformation under physiological conditions are expected to elute last. Therefore, if the portion collected has an aggregate number under physiological conditions which is greater than desired, the original β-glucan composition can be fractionated again, and a smaller trailing edge portion can be collected to obtain a β-glucan composition having a smaller aggregate number under physiological conditions. Preferably, the parameters are optimized using an analytical scale GPC column.

In a further embodiment, the present invention provides a method of forming a glucan composition comprising β-glucan chains which are in a triple helix aggregate conformation. The method comprises the steps of (1) reacting a highly branched β-glucan under conditions sufficient to remove at least a portion of the branches to form a debranched β-glucan and (2) maintaining the debranched β-glucan under conditions sufficient for formation of a triple helix aggregate form.

The highly branched β-glucan is a β-glucan which is substantially more branched than glucan, for example, a β-glucan which is too highly branched to form triple helix aggregates. For example, the highly branched β-glucan can be at least about 25% branched. In a preferred embodiment, the branches are joined to the main chain via β(1,6)-glycosidic bonds. Suitable examples of highly branched β-glucans of this type include scleroglucan, which is about 30-33% branched, schizophyllan, lentinan, cinerean, grifolan and pestalotan.

The highly branched β-glucan can be debranched by cleaving a portion of the bonds joining the branches to the main polymer chain. For example, when the branches are joined to the main polymer chain by β(1,6)-glycosidic bonds, the β(1,6)-glycosidic bonds can be hydrolyzed under conditions which leave the main polymer chain substantially intact. For example, hydrolysis of the β(1,6)-glycodsidic bonds can be catalyzed by an enzyme which preferentially cleaves β(1,6)-glycosidic bonds over β(1,3)-glycosidic bonds. Such enzymes of this type include hydrolases which are specific for or preferentially cleave β(1,6)-glycosidic bonds, for example, endoglycosidases, such as β(1,6)-glycosidases (Sasaki et al., Carbohydrate Res. 47: 99-104 (1976)).

The highly branched β-glucan can also be debranched using chemical methods. A preferred chemical debranching method is the Smith degradation (Whistler et al., Methods Carbohydrate Chem. 1: 47-50 (1962)). In this method the β-glucan is treated for about 3 days in the dark with a limiting amount of NaIO₄, based on the extent of debranching desired. The reaction is next quenched with ethylene glycol and dialyzed. The reaction mixture is then treated with excess NaBH₄, then quenched with acetic acid and dialyzed. The reaction mixture is then heated for about 3 hours at 80° C. with 0.2 M trifluoroacetic acid. The reaction mixture is then dialyzed and concentrated.

The debranching reaction is performed under conditions suitable for forming a glucan composition which is sufficiently debranched to permit triple helix aggregate formation. For example, in one embodiment, the extent of branching of the debranched β-glucan is less than about 10%. In a preferred embodiment, the debranched β-glucan is branched to substantially the same extent as PGG-glucan (about 7%).

Indications

The β-glucan compositions of the present invention have utility as safe, effective, therapeutic and/or prophylactic agents, either alone or as adjuvants, to enhance the immune response in humans and animals. An individual skilled in the medical arts will be able to determine the length of time during which the composition is administered and the dosage, depending on the physical condition of the patient and the disease or disorder being treated. As stated above, the composition may also be used as a preventative treatment to pre-initiate the normal metabolic defenses, which the body mobilizes against infections. The soluble β-glucan act without stimulating or priming the immune system to release certain biochemical mediators (e.g., IL-1, TNF, IL-6, IL-8 and GM-CSF) that can cause adverse side effects. As such, the present soluble glucan composition can be used to prevent or treat infectious diseases in malnourished patients, patients undergoing surgery and bone marrow transplants, patients undergoing chemotherapy or radiotherapy, neutropenic patients, HIV-infected patients, trauma patients, burn patients, patients with chronic or resistant infections such as those resulting from myelodysplastic syndrome, and the elderly, all of who may have weakened immune systems.

More particularly, the method of the invention can be used to therapeutically or prophylactically treat animals or humans who are at a heightened risk of infection due to imminent surgery, injury, illness, radiation or chemotherapy, or other condition, which deleteriously affects the immune system. The method is useful to treat patients who have a disease or disorder, which causes the normal metabolic immune response to be reduced or depressed, such as HIV infection (AIDS). For example, the method can be used to pre-initiate the metabolic immune response in patients who are undergoing chemotherapy or radiation therapy, or who are at a heightened risk for developing secondary infections or post-operative complications because of a disease, disorder or treatment resulting in a reduced ability to mobilize the body's normal metabolic responses to infection. Treatment with the soluble glucans has been shown to be particularly effective in mobilizing the host's normal immune defenses, thereby engendering a measure of protection from infection in the treated host.

β-glucan compositions can be used for the prevention and treatment of infections caused by a broad spectrum of bacterial, fungal, viral and protozoan pathogens. The prophylactic administration of β-glucan to a person undergoing surgery, either preoperatively, intraoperatively and/or post-operatively, will reduce the incidence and severity of post-operative infections in both normal and high-risk patients. For example, in patients undergoing surgical procedures that are classified as contaminated or potentially contaminated (e.g., gastrointestinal surgery, hysterectomy, cesarean section, transurethral prostatectomy) and in patients in whom infection at the operative site would present a serious risk (e.g., prosthetic arthroplasty, cardiovascular surgery), concurrent initial therapy with an appropriate antibacterial agent and the present β-glucan preparation will reduce the incidence and severity of infectious complications.

In patients who are immunosuppressed, not only by disease (e.g., cancer, AIDS) but by courses of chemotherapy and/or radiotherapy, the prophylactic administration of the β-glucan will reduce the incidence of infections caused by a broad spectrum of opportunistic pathogens including many unusual bacteria, fungi and viruses. Therapy using β-glucan has demonstrated a significant radio-protective effect with its ability to enhance and prolong macrophage function and regeneration and, as a result enhance resistance to microbial invasion and infection.

In high risk patients (e.g., over age 65, diabetics, patients having cancer, malnutrition, renal disease, emphysema, dehydration, restricted mobility, etc.) hospitalization frequently is associated with a high incidence of serious nosocomial infection. Treatment with β-glucan may be started empirically before catheterization, use of respirators, drainage tubes, intensive care units, prolonged hospitalizations, etc. to help prevent the infections that are commonly associated with these procedures. Concurrent therapy with antimicrobial agents and the β-glucan is indicated for the treatment of chronic, severe, refractory, complex and difficult to treat infections.

Another particular use of the compositions of this invention is for the treatment of myelodysplastic syndrome (MDS). MDS, frequently referred to as preleukemia syndrome, is a group of clonal hematopoietic stem cell disorders characterized by abnormal bone marrow differentiation and maturation leading to peripheral cytopenia with high probability of eventual leukemic conversion. Recurrent infection, hemorrhaging and terminal infection resulting in death typically accompany MDS. Thus, in order to reduce the severity of the disease and the frequency of infection, compositions comprising modified glucan can be chronically administered to a patient diagnosed as having MDS according to the methods of this invention, in order to specifically increase the infection fighting activity of the patient's white blood cells. Other bone marrow disorders, such as aplastic anemia (a condition of quantitatively reduced and defective hematopoiesis) can be treated to reduce infection and hemorrhage that are associated with this disease state.

The β-glucan compositions of the invention are also of use in methods of inducing or enhancing mobilization of peripheral blood precursor cells, elevating circulating levels of peripheral blood precursor cells and enhancing or facilitating hematopoietic reconstitution or engraftment in mammals, including humans. Peripheral blood precursor cells include stem cells and early progenitor cells which, although more differentiated than stem cells, have a greater potential for proliferation than stem cells. These methods comprise administering to the mammal an effective amount of a β-glucan composition of the present invention. Such methods are of use, for example, in the treatment of patients undergoing cytoreductive therapy, such as chemotherapy or radiation therapy.

β-glucan enhances the non-specific defenses of mammalian mononuclear cells and significantly increases their ability to respond to an infectious challenge. The unique property of β-glucan macrophage activation is that it does not result in increased body temperature (i.e., fever) as has been reported with many non-specific stimulants of those defenses. This critical advantage of β-glucan may lie in the natural profile of responses it mediates in white blood cells. It has been shown that the neutral soluble β-glucan of the present invention selectively activates immune responses but does not directly stimulate or prime proinflammatory cytokine (e.g., IL-1 and TNF) release from mononuclear cells, thus distinguishing the present β-glucan from other glucan preparations (e.g., lentinan, kresein) and immunostimulants.

In addition, it has been demonstrated herein that the β-glucan preparation of the present invention possesses an unexpected platelet stimulating property. Although it was known that glucans have the ability to stimulate white blood cell hematopoiesis, the disclosed platelet stimulating property had not been reported or anticipated. This property can be exploited in a therapeutic regimen for use as an adjuvant in parallel with radiation or chemotherapy treatment. Radiation and chemotherapy are known to result in neutropenia (reduced polymorphonuclear (PMN) leukocyte cell count) and thrombocytopenia (reduced platelet count). At present, these conditions are treated by the administration of colony stimulating factors such as GM-CSF and G-CSF. Such factors are effective in overcoming neutropenia, but fail to impact upon thrombocytopenia. Thus, the platelet stimulating property of β-glucans can be used, for example, as a therapeutic agent to prevent or minimize the development of thrombocytopenia which limits the dose of the radiation or chemotherapeutic agent which is used to treat cancer.

Administration

The present composition is generally administered to an animal or a human in an amount sufficient to produce immune system enhancement. The β-glucan portion of the combination composition of the invention can be administered parenterally by injection, e.g., subcutaneously, intravenously, intramuscularly, intraperitoneally, topically, orally or intranasaly. The soluble β-glucans may be administered as a solution having a concentration of from about 1 mg/ml to about 5 mg/ml. The solvent can be a physiologically acceptable aqueous medium, such as water, saline, PBS or a 5% dextrose solution. The amount necessary to induce immune system enhancement will vary on an individual basis and be based at least in part on consideration of the individual's size, the severity of the symptoms and the results sought. Soluble β-glucans may be administered into patients at doses up to about 10 mg/kg of patient weight.

The β-glucan portion of the composition of the invention is generally administered to an animal or a human in an amount sufficient to produce immune system enhancement. The mode of administration of the β-glucan can be oral, enteral, parenteral, intravenous, subcutaneous, intraperitoneal, intramuscular, topical or intranasal. The form in which the β-glucan will be administered (e.g., powder, tablet, capsule, solution, emulsion) will depend on the route by which it is administered. The quantity of β-glucan to be administered will be determined on an individual basis, and will be based at least in part on consideration of the severity of infection or injury in the patient, the patient's condition or overall health, the patient's weight and the time available before surgery, chemotherapy or other high-risk treatment. In general, a single dose will preferably contain approximately 0.01 to approximately 10 mg of modified glucan per kilogram of body weight, preferably from about 0.1 to 2.5 mg/kg and more preferably from about 0.25 to about 2 mg/kg. The dosage for topical application will depend upon the particular wound to be treated, the degree of infection and severity of the wound. A typical dosage for wounds will be from about 0.001 mg/ml to about 2 mg/ml, and preferably from about 0.01 to about 0.5 mg/ml.

In general, the composition of the present invention can be administered to an individual periodically as necessary to stimulate the individual's immune response. An individual skilled in the medical arts will be able to determine the length of time during which the composition is administered and the dosage, depending on the physical condition of the patient and the disease or disorder being treated. As stated above, the composition may also be used as a preventative treatment to pre-initiate the normal metabolic defenses, which the body mobilizes against infections.

The β-glucan portion of the compositions administered in the method of the present invention can optionally include other components, in addition to the neutral soluble β-glucans. The other components that can be included in a particular composition are determined primarily by the manner in which the composition is to be administered. For example, a composition to be administered orally in tablet form can include, in addition to neutral soluble β-glucan, a filler (e.g., lactose), a binder (e.g., carboxymethyl cellulose, gum arabic, gelatin), an adjuvant, a flavoring agent, a coloring agent and a coating material (e.g., wax or plasticizer). A β-glucan portion of the composition to be administered in liquid form can include neutral soluble β-glucan and, optionally, an emulsifying agent, a flavoring agent and/or a coloring agent. A β-glucan portion of the composition for parenteral administration can be mixed, dissolved or emulsified in water, sterile saline, phosphate buffered saline (PBS), dextrose or other biologically acceptable carrier. A composition for topical administration can be formulated into a gel, ointment, lotion, cream or other form in which the composition is capable of coating the site to be treated, e.g., wound site.

Embodiments of the invention described herein contemplate and encompass human antibodies. Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a mammal other than a rodent.

The ability to clone and reconstruct megabase-sized human loci in YACs and to introduce them into the mouse germline provides a powerful approach to elucidating the functional components of very large or crudely mapped loci as well as generating useful models of human disease. An important practical application of such a strategy is the “humanization” of the mouse humoral immune system. Introduction of human immunoglobulin (Ig) loci into mice in which the endogenous Ig genes have been inactivated offers the opportunity to develop human antibodies in the mouse. Fully human antibodies are expected to minimize the immunogenic and allergic responses intrinsic to mouse or mouse-derivatized monoclonal antibodies and thus to increase the efficacy and safety of the antibodies administered to humans. The use of fully human antibodies can be expected to provide a substantial advantage in the treatment of chronic and recurring human diseases, such as inflammation, autoimmunity, and cancer, which require repeated antibody administrations.

One approach toward this goal was to engineer mouse strains deficient in mouse antibody production with large fragments of the human Ig loci in anticipation that such mice would produce a large repertoire of human antibodies in the absence of mouse antibodies. This general strategy was demonstrated in connection with the generation of the first XenoMouse® strains as published in 1994. See Green et al., Nature Genetics 7:13-21 (1994).

Alternative approaches have utilized a “minilocus” approach, in which an exogenous Ig locus is mimicked through the inclusion of pieces (individual genes) from the Ig locus. Thus, one or more V_{H} genes, one or more DH genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

While chimeric antibodies have a human constant region and a murine variable region, it is expected that certain human anti-chimeric antibody (HACA) responses will be observed, particularly in chronic or multi-dose utilizations of the antibody.

Humanization and Display Technologies

Antibodies with reduced immunogenicity can be generated using humanization and library display techniques. It will be appreciated that antibodies can be humanized or primatized using techniques well known in the art. See e.g., Winter and Harris, Immunol Today 14:43-46 (1993) and Wright et al., Crit, Reviews in Immunol. 12:125-168 (1992). The antibody of interest can be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439 (1987) and J. Immuno 1.139:3521 (1987)). mRNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, an expression library is made and screened to isolate the sequence of interest encoding the variable region of the antibody is then fused to human constant region sequences. The sequences of human constant regions genes can be found in Kabat et al., “Sequences of Proteins of Immunological Interest,” N.I.H. publication no. 91-3242 (1991). Human C region genes are readily available from known clones. The choice of isotype will be guided by the desired effector functions, such as complement fixation, or activity in antibody-dependent cellular cytotoxicity. Preferred isotypes are IgG1, IgG2 and IgG4. Either of the human light chain constant regions, kappa or lambda, can be used. The chimeric, humanized antibody is then expressed by conventional methods. Expression vectors include plasmids, retroviruses, YACs, EBV derived episomes, and the like.

Antibody fragments, such as Fv, F(ab′)₂ and Fab can be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama et al., Mol. Cell. Bio. 3:280 (1983)), Rous sarcoma virus LTR (Gorman et al., P.N.A.S. 79:6777 (1982)), and moloney murine leukemia virus LTR (Grosschedl et al., Cell 41:885 (1985)). Also, as will be appreciated, native Ig promoters and the like can be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau, PNAS USA 94:4937-4942 (1997) (ribosomal display), Parmley and Smith, Gene 73:305-318 (1988) (phage display), Scott, TIBS 17:241-245 (1992), Cwirla et al., PNAS USA 87:6378-6382 (1990), Russel et al., Nucl. Acids Res. 21:1081-1085 (1993), Hoganboom et al., Immunol. Reviews 130:43-68 (1992), Chiswell and McCafferty, TIBTECH 10:80-84 (1992), and U.S. Pat. No. 5,733,743. If display technologies are utilized to produce antibodies that are not human, such antibodies can be humanized as described above.

Other Anti-EGF Receptor Antagonists

The anti-VEGF receptor antagonists used in the composition of the invention are not limited to bevacizumab, or even antibodies themselves. Monoclonal or polyclonal antibodies, antibody fragments or other proteins or small molecules are also contemplated by the invention. The one property these molecules must share is the ability to specifically bind to the VEGF receptor so as to block the interaction of VEGF with the receptor, thereby preventing mitogenic events associated with VEGF.

Specific molecules that may be used in the composition of the invention include the following. Monoclonal or polyclonal antibodies which bind to the VEGF receptor from various species including rat, mouse, horse, cow, goat, sheep, pig and rabbit are contemplated for use in the composition of the invention. Also, chimeric antibodies, produced from a human antibody and monoclonal antibodies made in any of the above mentioned animals are contemplated for use in the composition of the invention. Antibody fragments, especially variable regions from antibodies, which bind to VEGF-receptor are contemplated for use in the composition of the invention. Also, VEGF mutants which, while still able to bind to the VEGF receptor, do not cause signaling through the receptor and block antigenic signaling of wild-type VEGF are contemplated for use in the composition of the invention. Small molecules, which bind to VEGF receptor and block VEGF signaling through the receptor are also contemplated for use in the composition of the invention. Further, soluble VEGF receptor fragments, for example, encompassing the extracellular domain of the VEGF receptor, which are able to bind VEGF thereby preventing VEGF from binding to cell expressed wild-type VEGF receptor are also contemplated for use in the composition of the invention.

Indications

VEGF receptor antagonists are used to treat cancer. For example, bevacizumab has been FDA approved to treat some cancers. However, VEGF receptor antagonists may be found to effective against other cancers as well.

VEGF receptor antagonists may also be combined with other anti-cancer drugs for the treatment of cancer, for example, doxorubicin, cisplatin and irinotecan. Anti-cancer drugs are also contemplated as part of the combination composition of the invention. Other anti-cancer drugs, for example, include taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including creams, lotions, mouthwashes, inhalants and the like.

For human administration, preparations should meet sterility, pyrogenicity, general safety and purity standards as required by the FDA.

Administration of compounds alone or in combination therapies may be, e.g., subcutaneous, intramuscular or intravenous injection, or any other suitable route of administration. A particularly convenient frequency for the administration of the compounds of the invention is once a day.

Upon formulation, therapeutics will be administered in a manner compatible with the dosage formulation, and in such amount as is pharmacologically effective. The formulations are easily administered in a variety of dosage forms, such as the injectable solutions described, but drug release capsules and the like can also be employed. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

A carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Suitable preservatives for use in solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The compounds and combination therapies of the invention can be formulated by dissolving, suspending or emulsifying in an aqueous or nonaqueous solvent. Vegetable (e.g., sesame oil, peanut oil) or similar oils, synthetic aliphatic acid glycerides, esters of higher aliphatic acids and propylene glycol are examples of nonaqueous solvents. Aqueous solutions such as Hank's solution, Ringer's solution or physiological saline buffer can also be used. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for subcutaneous or intramuscular injection is also contemplated. In this regard, the use of DMSO as solvent is preferred as this will result in extremely rapid penetration, delivering high concentrations of the active compound(s) or agent(s) to a small area.

Where one or both active ingredients of the combination therapy are given orally, it can be formulated through combination with pharmaceutically acceptable carriers that are well known in the art. The carriers enable the compound to be formulated, for example, as a tablet, pill, capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

While this invention has been shown and described with references to particular embodiments, it will be understood by those skilled in the art that various changes in form and detail may be made therein without departing from the spirit and scope of the invention encompassed by the appended claims.

1. A therapeutic composition to treat a proliferative disorder, the composition comprising:
a VEGF antagonist; and
β-glucan.

2. The composition of claim 1, wherein the β-glucan is soluble, insoluble or a combination thereof.

3. The composition of claim 1, wherein the VEGF antagonist is an antibody.

4. The composition of claim 3, wherein the antibody is polyclonal.

5. The composition of claim 3, wherein the antibody is monoclonal.

6. The composition of claim 3, wherein the VEGF antagonist is a chimeric antibody.

7. The composition of claim 3, wherein the antibody is bevacizumab.

8. The composition of claim 1, further comprising an anti-cancer drug.

9. The composition of claim 8, wherein the anti-cancer drug is a member of the group consisting of ironotecan, doxorubicin and cisplatin.

10. The composition of claim 1, wherein the β-glucan is administered to the subject at a dose from about 0.1 to about 6 mg/kg.

11. A kit comprising a therapeutic dose of a β-glucan and a therapeutic dose of an VEGF antagonist either in the same or separate packaging, and instructions for its use.

12. The kit of claim 11, wherein the β-glucan is soluble, insoluble or a combination thereof.

13. The kit of claim 11, wherein the β-glucan is derived from yeast.

14. The kit of claim 11, wherein the VEGF antagonist is an antibody.

15. The kit of claim 14, wherein the antibody is polyclonal.

16. The kit of claim 14, wherein the antibody is monoclonal.

17. The kit of claim 14, wherein the antibody is bevacizumab.

18. A method of treating a proliferative disorder in a subject, the method comprising administering to the subject an effective amount of a β-glucan and an effective amount of an VEGF antagonist, thereby treating the proliferative disorder in the subject.

19. The method of claim 18, wherein the proliferative disorder is cancer.

20. The method of claim 19, further comprising administering an effective amount of an anti-cancer drug.
